(19) United States
(12) Patent Application Publication
(10) Pub. No.: US 2008/0281140 A1
(43) Pub. Date: Nov. 13, 2008

(54) Method and Device for Completely Hydrogenating a Hydrocarbon Flow

(75) Inventors: Falk Simon, Bensheim (DE); Sven Crone, Limburgerhof (DE); Goetz-Peter Schindler, Mannheim (DE); Peter Matthias Fritz, Muenchen (DE); Heinz Boelt, Wolfratshausen (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, LUDWIGSHAFEN (DE)

(21) Appl. No.: 11/719,726

(22) PCT Filed: Nov. 16, 2005
(86) PCT No.: PCT/EP05/12284

§ 371 Date: May 18, 2007

(30) Foreign Application Priority Data
Nov. 19, 2004 (DE) 10-2004-055-826.4

(51) Int. Cl. C07C 327/00; B01J 19/00

(52) U.S. Cl. 585/654; 422/189

(57) ABSTRACT

The invention provides a process for hydrogenating streams in plants for producing alkenes by catalytic dehydrogenation of light alkanes, and also an apparatus for carrying out the process. The entire hydrocarbon stream to the dehydrogenation reactor, consisting of fresh and recycled alkane, is subjected upstream of the dehydrogenation reactor to a full hydrogenation of all unsaturated hydrocarbons present therein. This drastically reduces coke formation in the dehydrogenation reactor. The energy demand for the preheating of the reactant stream to reaction temperature is reduced since the energy released in the exothermic hydrogenation remains virtually fully in the hydrocarbon stream.

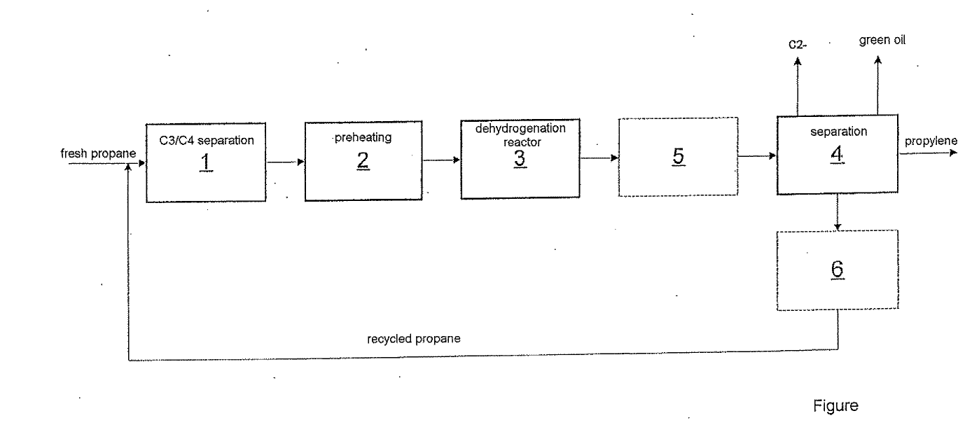

The invention provides a process for fully hydrogenating the hydrocarbon stream to the dehydrogenation reactor of plants for producing alkenes by catalytic dehydrogenation of light alkanes, and also an apparatus for carrying out the process.

Until a few decades ago, alkenes such as propylene and isobutene were obtained mainly as by-products in processes such as ethylene production in a steamcracker, for example. In these processes, however, certain alkenelethylene ratios cannot be exceeded. For propylene, this limiting value is, for example, approx. 0.65. Since, for example, the market for propylene has developed more strongly than the ethylene market in the last few decades, it has been necessary, in order to cover the rising demand, to find novel methods for the industrial scale production of this substance. In addition to alkene recovery from refinery cracking gas, a significant process has turned out to be dehydrogenation, i.e. the elimination of hydrogen, in which, for example, propene is obtained from propane and isobutene from isobutane in an economically viable manner.

In the last few years, several processes have been developed for the industrial dehydrogenation of light alkanes and some of them have been implemented on the industrial scale. These include the UOP Oleflex, Lummus Catofin, Linde PDH, Snamprogetti/Yarsintez FBD and Phillips STAR processes.

For all of their differences, the abovementioned processes have a common basic principle, as will be illustrated with reference to the FIGURE for the case of propane dehydrogenation, which is also valid for the dehydrogenation of other alkanes:

The feed of fresh propane is first purified in a C₃/C₄ separation stage (1) to remove heavier constituents (C₄⁺), as are always present as impurities, and, after a preheating (2) to reaction temperature, fed to a reactor (3) in which the catalytically promoted, endothermic dehydrogenation reaction proceeds. For thermodynamic and process technology reasons, between 50 and 70% of the propane leaves the reactor without chemical change (conversion). Nor does any of the processes possess 100% selectivity, i.e. from the propane molecules involved in the chemical reactions, a proportion (<20%) of other substances is also formed in addition to the desired propylene product (C₃H₈→C₃H₆+H₂). These include CH₄ and C₂H₄ which are formed by cracking reactions, acetylenes and diolefins (predominantly methylacetylene and propadiene) which are formed by double dehydrogenation, and green oil, which is a mixture of different long oligomers. This mixture is separated into fractions in several process steps (4). The light fractions (C₂⁻), green oil and propylene are discharged from the process; the propane is recycled and mixed with the freshly supplied propane upstream of the C₃/C₄ separation stage (1).

The deposition of coke deactivates the catalyst in the dehydrogenation reactor in the course of time and it has to be activated again by burning-off. The smaller the amount of coke which is deposited, the lower the level of complexity and expense for the activation. In addition to olefins, a particular contribution to coke formation is made by highly unsaturated components (for example acetylenes and diolefins) which are pre-sent in the hydrocarbon stream to the dehydrogenation reactor (fresh propane together with recyclate). A further negative aspect is a reduction in the propylene yield as a result of the reduction in the selectivity of the dehydrogenation reaction as a result of the unsaturated components in said hydrocarbon stream. For this reason, in the above-mentioned processes, before the propane recycling, the acetylenes and diolefins are converted by selective hydrogenation to propylene or by full hydrogenation to propane. The hydrogenation apparatus is integrated into the plant at different points. For example, in one embodiment of the Oleflex process, a selective hydrogenation is carried out in the liquid product stream beyond the dehydrogenation reactor (5), while, in another embodiment, the propane recyclate is subjected to a full hydrogenation (6). In the Linde-PDH process, a selective hydrogenation is effected in the bottom of the C3 splitter, i.e. likewise in the liquid phase (6), immediately before the propane recycling.

When the above-described methods are used for selective hydrogenation, small amounts of propylene are present in the propane recyclate and hence also in the hydrocarbon stream to the dehydrogenation reactor. Together with the unsaturated components which may be present in the fresh feed, this propylene always leads to the abovementioned adverse phenomena and thus to a reduction in the economic viability of the processes.

It is therefore an object of the present invention to configure a process of the type mentioned at the outset and also an apparatus for carrying out the process in such a way that all unsaturated components in the hydrocarbon stream to the dehydrogenation reactor of plants for dehydrogenating light alkanes are removed in an economically viable manner.

In process terms, this object is achieved in accordance with the invention by carrying out a full hydrogenation of all unsaturated components in the entire hydrocarbon stream flowing to the dehydrogenation reactor.

With reference to the basic principle illustrated in the FIGURE, the full hydrogenation of the hydrocarbon stream to the dehydrogenation reactor (fresh and recycled alkane) is carried out in the gas phase preferably after the separation stage (1) and before the preheating (2). For this purpose, hydrogen is admixed to the hydrocarbon stream before it is conducted over a suitable catalyst. In the catalyst bed, the exothermic hydrogenation reaction proceeds in such a way that, when it enters the preheating stage, virtually exclusively alkane and excess hydrogen are present in the hydrocarbon stream.

Preference is given in accordance with the invention to carrying out the full hydrogenation generally not under stoichiometric conditions, but rather with hydrogen excess. As a result, the hydrogenation, even without regulation of the amount of hydrogen, can proceed to completion at any time with changing operating conditions. When the content of unsaturated components in the hydrocarbon stream rises, the energy released in the hydrogenation increases. Since the hydrocarbon stream has to be preheated up to reaction temperature in any case, this effect is not disadvantageous.

Excess hydrogen is not removed from the hydrocarbon stream. A decline in the content of unsaturated components therefore has the consequence of stronger hydrogen dilution of the hydrocarbon stream, which leads, however, to higher selectivity and to suppression of green oil formation. In addition, the hydrogen excess prolongs the running time of the hydrogenation reactor.

The energy released in the exothermic full hydrogenation can be utilized directly to pre-heat the hydrocarbon stream to the dehydrogenation reactor (3). Thus, the energy demand in the preheating stage (2) of the plant falls. For this reason, the hydrocarbon stream is appropriately not cooled at the outlet of the hydrogenation reactor; instead, the hydrogenation is preferably carried out under adiabatic conditions. As a result, virtually all of the energy released is kept within the stream.

The invention further relates to an apparatus for hydrogenating the hydrocarbon stream to the dehydrogenation reactor of plants for alkene production by catalytic dehydrogenation of light alkanes.

In apparatus terms, the object of the invention is achieved by, in a unit disposed preferably downstream of the separation stage (1) and upstream of the preheating stage (2), the entire hydrocarbon stream flowing to the dehydrogenation reactor being subjected to a full hydrogenation of all unsaturated components present therein.

Preference is given to providing the unit for fully hydrogenating the hydrocarbon stream with a hydrogen supply which enables the hydrogen stream to be adjusted in such a way that the hydrogenation is carried out under all operating conditions with hydrogen excess, in the extreme case under stoichiometric conditions, but never with hydrogen deficiency.

The hydrogenation reactor is appropriately designed as an adiabatic reactor, i.e. the reactor is not equipped with a unit which removes energy being released during the endothermic reaction. Instead, the reactor is appropriately provided with heat insulation which ensures that the energy being released remains virtually fully in the hydrocarbon stream. The hydrogenation energy contributes partly to the preheating of the reactant stream to the reaction temperature needed for the dehydrogenation.

Because the energy demand in the actual preheating stage is lower in comparison to the prior art as a result, the latter can be designed in a smaller and less expensive manner.

As a result of the use of the inventive apparatus, it is possible to dispense with reactors for selectively hydrogenating or for fully hydrogenating the recyclate, as are state of the art, for example, in industrial scale plants for propane dehydrogenation.

1-9. (canceled)

10. A process for fully hydrogenating a hydrocarbon stream flowing to a dehydrogenation reactor used for producing alkenes via catalytic dehydrogenation of light alkanes, said process comprising the step of fully hydrogenating all unsaturated components in said hydrocarbon stream.

11. The process according to claim 10, wherein said step is carried out with a stoichiometric amount or an excess of hydrogen.

12. The process according to claim 10, wherein said step is carried out in the gas phase.

13. The process according to claim 11, wherein said step is carried out in the gas phase.

14. The process according to claim 10, wherein energy released as a result of said step is substantially kept within the hydrogenated stream and partially preheats said hydrogenated stream to dehydrogenation reaction temperature.

15. The process according to claim 11, wherein energy released as a result of said step is substantially kept within the hydrogenated stream and partially preheats said hydrogenated stream to dehydrogenation reaction temperature.

16. The process according to claim 12, wherein energy released as a result of said step is substantially kept within the hydrogenated stream and partially preheats said hydrogenated stream to dehydrogenation reaction temperature.

17. The process according to claim 10, wherein said light alkane is propane and said alkene is propylene.

18. The process according to claim 11, wherein said light alkane is propane and said alkene is propylene.

19. The process according to claim 12, wherein said light alkane is propane and said alkene is propylene.

20. The process according to claim 13, wherein said light alkane is propane and said alkene is propylene.

21. An apparatus for fully hydrogenating a hydrocarbon stream flowing to a dehydrogenation reactor used for producing alkenes via catalytic dehydrogenation of light alkanes, said apparatus comprising a unit disposed upstream of the dehydrogenation reactor, wherein all unsaturated components present in said hydrocarbon stream are fully hydrogenated in said unit.

22. The apparatus according to claim 21, wherein said unit comprises an adjustable hydrogen supply which is adjusted so that, even in the event of changing operating conditions, full hydrogenation of all unsaturated components present in said hydrocarbon stream is carried out with a stoichiometric amount or an excess of hydrogen.

23. The apparatus according to claim 21, wherein said unit is configured as an adiabatic reactor.

24. The apparatus according to claim 22, wherein said unit is configured as an adiabatic reactor.

25. The apparatus according to claim 21, wherein said unit is designed for fully hydrogenating a gaseous hydrocarbon stream.

26. The apparatus according to claim 22, wherein said unit is designed for fully hydrogenating a gaseous hydrocarbon stream.

27. The apparatus according to claim 23, wherein said unit is designed for fully hydrogenating a gaseous hydrocarbon stream.
